(19) United States
(12) Patent Application Publication
(10) Pub. No.: US 2004/0058867 A1
(43) Pub. Date: Mar. 25, 2004

(54) Management of the consequences of fetal programming

(76) Inventors: Mark Hedley Vickers, Auckland (NZ); Bernhard Hermann Heinrich Breier, Auckland (NZ); Bettina Anastasia Ikenasio, Auckland (NZ)

Correspondence Address: Sheldon R Meyer, Fliesler Dubb Meyer and Lovejoy, Suite 400, Four Embarcadero Center, San Francisco, CA, 94111-4156, US

(21) Appl. No.: 10/450,232

(22) PCT Filed: Dec. 11, 2001
(86) PCT No.: PCT/NZ01/00277

(30) Foreign Application Priority Data
Dec. 11, 2000 (NZ) 508779

(51) Int. Cl. A61K 38/30

(52) U.S. Cl. 514/12

(57) ABSTRACT

A method of ameliorating or preventing the consequences of fetal programming in an otherwise normal mammal, including the administration to the mammal of an effective amount of insulin-like growth factor (IGF-I), an analogue thereof, or a functionally equivalent ligand.

### FIELD OF THE INVENTION

This invention relates to insulin-like growth factor-1(IGF-I) and its application for the management of metabolic disorders or other physiological disorders which can result from fetal programming.

### BACKGROUND

There is increasing evidence that metabolic disorders which manifest in adult life have their roots before birth. This concept of fetal programming is based on epidemiological and experimental observations of close associations between an adverse intrauterine environment and the later onset of adult metabolic and cardiovascular disorders. “Fetal programming” is herein defined as an adaptive process to an adverse intrauterine environment which alters the fetal metabolic and hormonal milieu, resulting in resetting of developmental processes to ensure fetal survival. The persistence of these adaptive responses, designed for survival in a fetal environment, into postnatal life, leads to metabolic and cardiovascular disorders.

We have developed an animal model of fetal programming where we apply maternal undernutrition throughout gestation, generating a nutrient-deprived intrauterine environment that results in fetal growth retardation, postnatal growth failure and to changes in allometric growth patterns and endocrine parameters of the somatotrophic axis (1, 2). We have recently shown in our animal model that programmed offspring show profound hyperphagia and obesity, hypertension, hyperinsulinism and hyperleptinemia during adult life and that postnatal hypercaloric nutrition amplifies the metabolic and cardiovascular abnormalities induced by fetal programming (3). Thus, this animal model closely resembles the clinical and metabolic abnormalities seen in humans born of low birth weight and furthermore, displays the phenotype described for the clinical association between hypertension, hyperinsulinemia, dyslipidemia, obesity, and cardiovascular disease, known as Syndrome X. Epidemiological studies have shown that those born of low birth weight have increased rates of obesity in adult life (4). This was most clearly shown in a recent report from the Dutch Famine Study where poor nutrition in the first trimester of pregnancy resulted in increased rates of obesity during adult life (5). Animal studies have also shown that maternal malnutrition during pregnancy results in the development of adult-onset obesity in offspring (4, 6, 7).

Profound hyperphagia is a consequence of programming and a key contributing factor in adult pathogenesis. Food intake in programmed offspring is significantly elevated at an early postnatal age and increases further with advancing age (3). Our studies also suggest that an adverse intrauterine environment can trigger permanent dysregulation of endocrine systems that regulate food intake and energy homeostasis leading to increased adiposity, hypertension, hyperinsulinism and hyperleptinernia.

Insulin-like growth factor-I (IGF-I) is one of the most important regulators of growth and IGF-I deficiency is associated with prenatal and postnatal growth failure (8, 9). Under conditions of adequate nutrition, IGF-I has been shown to promote postnatal catch-up growth in rats with intrauterine growth retardation (IUGR) caused by gestational protein deficiency (10). IGF-I therapy is associated with increased insulin sensitivity in normal subjects as well as in patients with growth hormone deficiency, type 2 diabetes mellitus and type A insulin-resistance (11). IGF-I can reduce hyperglycemia in patients with severe insulin resistance by direct effects mediated via the IGF-I receptor (12). Hyperglycemia, hyperinsulinemia, and insulin resistance cause vascular disease in type 2 diabetes. IGF-I infusions lower insulin and lipid levels in healthy humans, and reduces plasma leptin concentrations in rats (13), suggesting that IGF-I may reduce the degree of insulin resistance in type 2 diabetes, obesity and hyperlipidemia (14). However, little is known about the effect of IGF-I on appetite. Infusion of IGF-I has been shown to reduce appetite in tumour-bearing rats (15) but a recent study showed no effect on food intake following IGF-I treatment in normal rats, despite the plasma leptin-lowering effects of IGF-I in that study (13).

Clinical studies relating to IGF-I in hypertension are limited but IGF-I has previously been shown to have vasodilatory effects and to improve cardiac finction in healthy volunteers (16). Animal studies suggest a role for IGF-I as a mediator of cardiac hypertrophic responses (17).

The effects of IGF-I on cardiovascular and metabolic homeostasis may be mediated by the insulin-like growth factor binding proteins (IGFBPs). IGFBP-1 and 2 levels closely reflect changes related to nutrition, insulin secretion and disease states such as obesity and type 2 diabetes. IGFBP-3 correlates with IGF-I and is a chronic indicator of GH-dependent growth status (18) while IGFBPs appears to inhibit IGF actions under most, if not all, experimental conditions (19). Previous work (1, 20, 21) has shown differential expression of IGFBPs following fetal growth retardation. However, to date, there are no data on the effect of IGF-I treatment on IGFBPs in postnatal life following fetal programming alone or in combination with hypercaloric nutrition.

It is an object of the present invention to provide a method of managing and/or preventing the development of metabolic disorders following fetal programming. It is a further or alternative object of the invention to provide a method or treatment for managing the consequences of fetal programming which reduces or overcomes at least some of the above mentioned problems, or which will at least provides the public with a useful alternative.

Other objects of the invention may become apparent from the following description which is given by way of example only.

### SUMMARY OF THE INVENTION

According to one aspect of the present invention there is provided a method of ameliorating or preventing the consequences of fetal programming in an otherwise normal mammal, including the administration to the mammal of an effective amount of insulin-like growth factor (IGF-I), an analogue thereof, or a finctionally equivalent ligand.

According to a further aspect of the present invention there is provided a method of ameliorating or preventing the consequences of fetal programming in a mammal, including the steps of:

identifying a mammal exposed to fetal programming, and

treating said mammal with an effective amount of IGF-I, an analogue thereof, or a functionally equivalent ligand.

In one preferred form the mammal is a human.

Preferably, the mammal has no physiological symptoms and/or outward signs resulting from the fetal programming.

Preferably, the mammal exposed to fetal programming is identified from a review of maternal history during pregnancy.

Preferably the method includes administration of a replicable vehicle encoding the IGF-1/ligand/analogue.

Preferably the replicable vehicle is administered in an implant.

Other aspects of the invention may become apparent from the following description, given by way of example and with reference to the experimental data.

### BRIEF DESCRIPTION OF THE FIGURES

FIG. 1.

Postnatal growth curves of AD and UN offspring from weaning until commencement of IGF-I treatment (AD control diet (open circles), AD hypercaloric diet (filled triangles), UN control diet (open squares), UN hypercaloric diet (filled diamonds). n=6 per group, data are mean ±SEM.

FIG. 2.

Weight gain (grams per day) during 14 days of IGF-I treatment. Programming effect NS, IGF-I treatment effect p<0.0001, diet effect p<0.05, diet×IGF-I treatment interaction p<0.05. n=6 per group, data are mean ±SEM.

FIG. 3.

Food intake (kcal consumed per gram body weight per day) during 14 days of IGF-I treatment. Programming effect p<0.0005, IGF-I treatment effect p<0.0001, diet effect p<0.0001, programming×IGF-I treatment interaction p<0.005, programming×IGF-I treatment×diet interaction p<0.05. n=6 per group, data are mean ±SEM.

FIG. 4

Change in systolic blood pressure after 14 days of IGF-I treatment. Programming effect p<0.0005, IGF-I effect p<0.005, diet effect NS. There were no significant statistical interactions. n=6 per group, data are mean ±SEM.

FIG. 5.

Blood plasma IGF-I concentrations. Programming effect NS, IGF-I treatment effect p<0.0001, diet effect NS, programming×IGF-I treatment interaction p<0.05. n=6 per group, data are mean ±SEM.

FIG. 6.

Fasting blood plasma insulin and glucose concentrations following 14 days IGF-I treatment. Insulin: programming effect p<0.05, IGF-I treatment effect p<0.0001, diet effect p<0.0005, diet×IGF-I treatment interaction p<0.0005. Glucose: programming effect NS, IGF-I treatment effect p<0.0001, diet effect p<0.0001. There were no significant statistical interactions for fasting plasma glucose concentrations. n=6 per group, data are mean ±SEM.

FIG. 7

Retroperitoneal and gonadal fat pad weight (expressed as percent body weight), plasma leptin concentrations following 14 days saline (open bars) or IGF-I (closed bars) treatment and the relationship between adipose mass and plasma leptin concentrations. Retroperitoneal fat: programming effect p<0.05, IGF-I treatment effect p<0.0001, diet effect p<0.0001. Gonadal fat: programming effect p<0.0001, IGF-I treatment effect p<0.0001, diet effect p<0.0001. Plasma leptin: programming effect p<0.005, IGF-I treatment effect p<0.0001, diet effect p<0.0005, programming×diet interaction p<0.05, diet×IGF-I interaction p<0.005. There were no significant statistical interactions for retroperitoneal and gonadal fat pad weight. n=6 per group, data are mean ±SEM.

FIG. 8.

Serum IGFBPs as quantified following ligand blotting analysis. IGFBP-3 (38-44 kDa): programming effect NS, IGF-I treatment effect p<0.0001, diet effect p<0.0001, programming×IGF-I treatment interaction p<0.0001, diet×IGF-I treatment interaction p<0.005, programming×IGF-I treatment×diet interaction p<0.05. IGFBP-1, -2 (28-30 kDa): programming effect NS, IGF-I treatment effect p<0.0001, diet effect p<0.05, pro gramming×IGF-I treatment interaction p<0.05. IGFBP-4 (24 kDa): programming effect p<0.0001, IGF-I treatment effect p<0.0001, diet effect p<0.0005, programming×IGF-I treatment interaction p<0.005, diet×IGF-I treatment interaction p<0.05. 38 kDa IGFBP-3: programming effect p<0.0001, IGF-I treatment effect p<0.0001, diet effect p<0.0005. There were no significant statistical interactions for the 38 kDa IGFBP-3 band. Sample was 2 μl, n=6 per group, data are mean ±SEM.

### DETAILED DESCRIPTION OF THE INVENTION

An animal model has been established which demonstrates the significance of fetal programming on the subsequent development of metabolic disorders in adult life. This model involves “fetal programming” whereby under nutrition of the mother during gestation leads to programming of hyperphagia, obesity, insulin resistance and hypertension in the offspring (hereafter referred to as “programming” or “programmed”). The model is described later herein under then heading “Experimental Data” and substantially mimics the metabolic syndrome in humans known as “Syndrome X”. Animal models are well known predictive tools that are used as they closely mimic the human condition. Such techniques are well known and are acceptable to those skilled in this art.

From this model it can be seen that many harmful conditions manifest in individuals due to the condition being brought about by a programming of the fetus to get those conditions later in life following birth. Once having noted this connection, treatment on a prophylactic basis and/or on a symptomatic basis can be proceeded with. Clearly, if a prophylactic treatment can be used that will lower or delay the incidence of the listed conditions in individuals, that would be very advantageous. This connection between fetal programming and onset of any one or more of a number of harmful conditions represents a significant advance allowing treatment programs for those conditions to be developed.

Physical or metabolic indicators that can be used to identify an individual at risk include, but are not necessarily limited to, maternal food deprivation, placental dysfunction, uteroplacental blood supply problems, intrauterine growth retardation (usually as a result of the previously listed conditions) altered levels of IGF-I and also evidence that the mother herself was exposed to “fetal programming” whilst in the womb (also known as “intergenerational effect”).

Children of lower birth weight can develop higher circulating concentrations of IGF-1 than expected for their height and weight. Undernutrition in utero can also lead to reprogramming of the IGF-1 axis. Increases in plasma IGF-1 concentrations in low birth weight children may be linked to post-natal catch-up growth. Hence, altered IGF-1 levels can be a useful indicator.

Evidence that the mother was herself exposed to fetal programmung while in the womb (the intergenerational effect”) can also be an indicator. If a mother's development was affected, the health of her fetus may be also.

Assessment of individuals on the basis of any one or more of the above indicators can be used to form the basis for a treatment regime to treat the consequences of fetal programming as is discussed herein.

The effects of IGF therapy on programmed animals receiving an ordinary or a hypercaloric diet postnatally, were investigated. IGF-I treatment markedly reduced appetite, obesity, hyperinsulinemia, hyperleptinemia and hypertension in these programmed animals. Importantly, there was no significant effect on blood pressure in normotensive aninals. The effects of IGF-I may involve restoration of a functional feedback between insulin and leptin and/or differential regulation of the insulin receptor substrate (IRS), renin-angiotensin system (RAS) and/or IGF-I receptor signalling pathways, perhaps via a differential effect on IGFBPs, although further work on this hypothesis is needed.

The effects of IGF-I shown in the animal model suggest that this substance also has value as a treatment for hyperinsulinemia or insulin resistance in subjects exposed to fetal programming, or indeed in those at risk of developing such conditions through fetal programning. In particular, it may have benefit in such subjects prior to the development of any outward or physiological symptoms.

Furthermore, IGF-I treatment may be of value in ameliorating or preventing the consequences of fetal programming in otherwise normal subjects. Such subjects would be selected according to their risk of developing hypertension, obesity, diabetes or other metabolic disorders as a consequence of exposure to particular conditions in utero (ie, through programming).

As a result the present invention provides a means of prophylactic treatment of the conditions resulting from fetal programming. Treatment may then continue to provide a therapeutic benefit for mammals showing symptoms of such conditions.

Subjects are selected for prophylactic treatment on the basis of a review of maternal history (as discussed above) during pregnancy. However, it will be appreciated that other indicators for selection of subjects for treatment may be identified, including particular metabolic indicators or a particular combination of metabolic indicators.

The early identification of programmed individuals who are otherwise normal healthy subjects, before they show any physiological signs of metabolic disturbance, enables effective management and prevention of the onset of hypertension, obesity, diabetes and other metabolic disorders; disorders which can be an enormous financial burden, with lifetime treatment. It may be that early treatment of such individuals will only delay onset of symptoms, however even such a delay is beneficial both in terms of an individual's quality of life and in terms of financial issues.

It is envisaged that the principal application of the methods of the present invention will be to humans, either as adults or juveniles, although the method may also have application to non-human mammals.

Whilst the method may involve administering an effective amount of IGF-I, it may alternatively use an analogue thereof or a functionally equivalent ligand that will bind to the IGF-1 receptors. The IGF-I can be any mammalian IGF-I, with examples being human IGP-I, porcine IGF-I, or ovine IGF-I and bovine IGF-I. It is, however, preferred that the IGF-I used be human IGF-I where the mammal is a human. In addition to IGF-I itself, the use of analogues of IGF-I or functionally equivalent ligands is contemplated. It will be appreciated that these benefits may be derived from the administration of ligands which bind to the IGF-I receptor as well as to IGF-I itself.

By analogues of IGF-I is meant compounds which exert a similar biological effect to IGF-I and includes IGF-2 and analogues of IGF-2 naturally occurring analogues (eg. des(1-3) IGF-I) or any of the known synthetic analogues of IGF-I. IGF-I and analogues can be purified from natural sources or produced by recombinant DNA techniques. Recombinant IGF-I and des(1-3) IGF-I can be obtained commercially.

As used herein, “analogue” means a protein which is a variant of IGF-I through insertion, deletion or substitution of one or more amino acids, but which retains at least substantial functional equivalency.

The term “functionally equivalent ligand” means an agent which binds to and activates the receptors which IGF-I binds to and activates to give the required effect.

A protein is a functional equivalent of another protein for a specific function if the equivalent protein is immunologically cross-reactive with, and has at least substantially the same function, as the original protein. The equivalent can be, for example, a fragment of the protein, a fusion of the protein with another protein or carrier, or a fusion of a fragment with additional amino acids. For example, it is possible to substitute amino acids in a sequence with equivalent amino acids using conventional techniques. Groups of amino acids normally held to be the equivalent are:

(a) Ala, Ser, Thr, Pro, Gly;

(b) Asn, Asp, Glu, Gln;

(c) His, Arg, Lys;

(d) Met, Leu, Ile, Val; and

(e) Phe, Tyr, Trp.

The present invention may also extend to the administration of an agent which either stimulates the production of IGF-I, or which lessens or prevents inhibition of IGF-I activity. There is a wide variety of biological compounds that exert a stimulatory or inhibitory effect over IGF-1. Growth hormone, estrogen and thyroid hormone have all been reported to have stimulatory effects. There are also patents for design of IGF analogues (example U.S. Pat. No. 6,251,865) that inhibit binding to IGF binding proteins, and for IGF inhibitors (example U.S. Pat. No. 6,121,416) that prevent binding of IGF-1 to its receptor. Such options could also be used in the present invention.

The active agent can be administered using any suitable route. Where IGF-I is the active agent, it may for example be administered orally or parenterally, in combination with one or more suitable carriers or excipients. Preferably the IGF-1 is dissolved in sterile saline or water. The preferred administration route is subcutaneous injection.

Another possibility is administration to the mammal of a replicable vehicle encoding the IGF-I/analogue/ligand. Such a vehicle (which may be a modified cell line or virus which expresses IGF-I/analogue/ligand within the mammal) could have application in increasing the concentration of the active compound within the mammal for a prolonged period. Such a vehicle could form part of an implant.

The present invention can therefore be seen to provide the use of an effective amount of IGF-1/analogue/ligand in the manufacture of a medicament for prevention of the onset of conditions that may result from fetal programming. Such conditions have been discussed previously herein. The actual manufacturing methods can be those known to the skilled person. Recognition of the likelihood of symptom onset being the key feature.

Dosage levels will be formulation dependent due to volume load. The amount of IGF-1 that can be administered will depend on the method of delivery. However, a suitable dosage range of IGF-I or analogues formulated for injection may be in the range of 0.1 μg/kg/day to 1 mg/kg/day. A preferred dosage rate would be from about 2 to 200 μg/kg/day.

Dosages from 40 to 80 μg/kg.day, by subcutaneous injection once or twice daily, and continued for 2-5 years or more, may be appropriate in children or young adults.

Experimental Data

Materials and Methods

Virgin Wistar rats (age 100±5 days, n=15 per group) were time mated using a rat oestrous cycle monitor to assess the stage of oestrous of the animals prior to introducing the male. After confirmation of mating, rats were housed individually in standard rat cages containing wood shavings as bedding and free access to water. All rats were kept in the same room with a constant temperature maintained at 25° C. and a 12-h light:12-h darkness cycle.

Animals were assigned to one of two nutritional groups: Group 1; undernutrition (30% of ad-libitum (UN)) of a standard diet throughout gestation, Group 2; standard diet AD throughout pregnancy. Food intake and maternal weights were recorded daily until birth. After birth, pups were weighed and litter size recorded. Pups from undernourished mothers were cross-fostered onto dams which received AD feeding throughout pregnancy. Litter size was adjusted to 8 pups per litter to assure adequate and standardised nutrition until weaning. After weaning, female offspring from the two groups of dams a) AD offspring and b) offspring from undernourished mothers (UN) were divided into 2 balanced postnatal nutritional groups to be fed either a standard diet (total digestible energy 2959 kcal/kg, protein 19.4%, fat 5%, fat/energy ratio 15.21%, protein energy ratio 26.23) or a hypercaloric diet; (total digestible energy 4846 kcal/kg, protein 31.8%, fat 30%, fat/energy ratio 55.72%, protein/energy ratio 26.25%). The mineral and vitamin content in the two diets were identical and in accordance with the requirements for standard rat diets. The fat content of the hypercaloric diet is typical of that seen in many Western diets. Weights and food intake of all offspring were measured daily for the first 2 weeks then every second day. At day 175, systolic blood pressure measurements were recorded using tail cuff plethysmography. Rats were then weight matched and received either rh-IGF-I (3 μg/g/day) or saline by osmotic minipump (Model 2002, Alzet Corp, Palo Alto, Calif. US) for 14 days. On the day prior to sacrifice, a repeated systolic blood pressure was recorded. Rats were then fasted overnight and sacrificed by halothane anaesthesia followed by decapitation. Blood was collected into heparinised vacutainers and stored on ice until centrifugation and removal of supernatant for analysis. All animal work was approved by the Animal Ethics Committee of the University of Auckland.

Blood Pressure Measurements Systolic blood pressure (SBP) was recorded by tail cuff plethysmography according to the manufacturers instructions (Blood pressure analyser IITC, Life Science, Woodland Hills, Calif., USA). Rats were restrained in a clear plastic tube in a heated room (25-28° C.). After the rats had acclimatised (10-15 min) the cuff was placed on the tail and inflated to 240 mmHg. Pulses were recorded during deflation at a rate of 3 mmHg/sec and reappearance of a pulse was used to determine systolic blood pressure. A minimum of three clear SBP recordings were taken per animal and the coefficient of variation for repeated measurements was <5%.

IGF-I Infusion

At day 175, rats were weight matched (n=6 per group) and received either rh-IGF-I (Genentech Code #G117AZ, Batch c9831AY) or saline by osmotic minipump (Model 2002, Alzet Corp, Palo Alto, Calif. US). The dose was 3 μg/g/day for 14 days with a pump delivery rate of 5 μl per hour. The mean pump rate for the batch (Lot # 167258) of pumps used was 5.23±0.2 μl/hr. Pumps containing the IGF-I or saline solution were incubated in sterile saline for 4 hours at 37° C. prior to implantation. The osmotic pumps were implanted subcutaneously, under halothane anesthesia, using a small incision made in the skin between the scapulae. Using a haemostat, a small pocket was formed by spreading apart the subcutaneous connective tissues. The pump was inserted into the pocket with the flow moderator pointing away from the incision. The skin incision was then closed with sutures. All animals (n=48) were housed individually for the duration of the study.

Radioimmunoassay (RIA) for Rat Insulin-Like Growth Factor-I (IGF-I)

IGF-I in rat blood plasma was measured using a IGF binding protein (IGFBP) blocked RIA described previously (22). The half maximally effective dose, or ED-50, was 0.1 ng/tube and the intra- and inter-assay coefficients of variation were <5% and <10% respectively.

RIA for Rat Insulin

Rat insulin was measured by RIA as described previously (3). Blood plasma was diluted 1:4 in assay buffer (0.01M PBS containing 0.37% Na EDTA and 0.5% BSA, pH 6.2). In brief, the primary antibody, (guinea-pig anti-ovine-Insulin) was diluted in assay buffer to an initial working dilution of 1:80000. 0.1 ml of diluted sample, control, or standard (rat insulin, 0.01-10 ng/ml, Crystal Chem., Chicago) .was incubated with 0.2 ml of primary antibody for 24 hours at room temperature. 0.2 ml ¹²⁵I-rh-Insulin (Eli Lily, Lot No 615-707-208) was then added at 15-20000 counts per tube. Equilibrium conditions were established after 24 hours incubation at 4° C. A second antibody was used to separate bound from free ligand as outlined previously (23) and the pellet counted by gamma counter. Rat plasma samples showed parallel displacement to the standard curve and recovery of unlabelled rat insulin was 96.5±4.4% (mean ±SEM, n=11). The half-maximally effective dose (ED-50) was 0.5 ng/ml.

RIA for Rat Leptin

A double antibody RIA was developed and validated for measurement of leptin in rat plasma An antibody was raised in rabbits against a fragment (aa 30-45) of bovine leptin. Standard preparation was rm-leptin (Crystal Chem, US., #CR-6781) used in concentrations ranging from 0.5 to 20 ng/ml. Samples were assayed neat or diluted 1:2-1:4 in assay buffer (0.05M PBS, pH 7.4 containing 0.1M NaCl, 0.5% BSA, 10 mM EDTA, 0.05% NaN₃). In brief, 100 μl of primary antibody (1:25000) was added to tubes containing 100 ul of sample or standard. Following incubation for 24 h at 4° C., 100 μl of tracer (¹²⁵I-rm-leptin, 20000 cpm per tube) was added to all tubes followed by a further incubation for 24 h at 4° C. A second antibody technique to separate bound from free ligand was used as outlined previously (23). Rat plasma samples showed parallel displacement to the standard curve and recovery of unlabelled rm-leptin was 101.4±2.7% (mean ±SEM, n=26). The ED-50 was 0.37 ng/ml and the intra-assay coefficient of variation was <5% (all samples measured within a single assay).

Blood Biochemistry

Plasma glucose concentrations were measured using a YSI Glucose Analyzer (Model 2300, Yellow Springs Instrument Co., Yellow Springs, Ohio, US). Blood plasma free fatty acids were measured by diagnostic kit (Boehringer-Mannheim #1383175). All other plasma analytes were measured by a BM/Hitachi 737 analyser by Auckland Healthcare Laboratory Services.

Ligand Blotting of Rat Plasma IGFBPs

IGFBPs in rat plasma (2 μl sample, n=6 per treatment group) were analyzed by ligand blotting (24) as described in detail elsewhere (25). Rat ¹²⁵I-IGF-II was used as radiolabel. Nitrocellulose blots were air dried and exposed to Kodak X-Omat AR diagnostic film (Eastman Kodak, Rochester, N.Y., USA) in Amersham Hyperscreen cassettes with intensifier screens. For quantification, nitrocellulose blots were exposed overnight to phospor imaging screens and analysed on a Storm PhosporImager system using ImageQuant software (Molecular Dynamics, Sky Valley, Calif., USA). All values were expressed relative to a normal rat plasma pool and standardised to 100% for control group. The IGFBPs were identified on the basis of their molecular size using nomenclature previously described (26).

Statistical Analysis

Statistical analyses were carried out using SigmaStat™ (Jandel Scientific, San Rafael, Calif., USA) and StatView™ (SAS Institute Inc., NS, USA) statistical packages. Differences between groups were determined by two-way (pre-IGF-I treatment) or three-way ANOVA (post-IGF-I treatment) followed by Bonferonni post-hoc analysis and data are shown as mean ±SEM. Plasma leptin and food intake data were also analysed by ANCOVA using unadjusted fat pad weight and body weights as covariates respectively. Statistical significance was assumed at the p<0.05 level.

Results

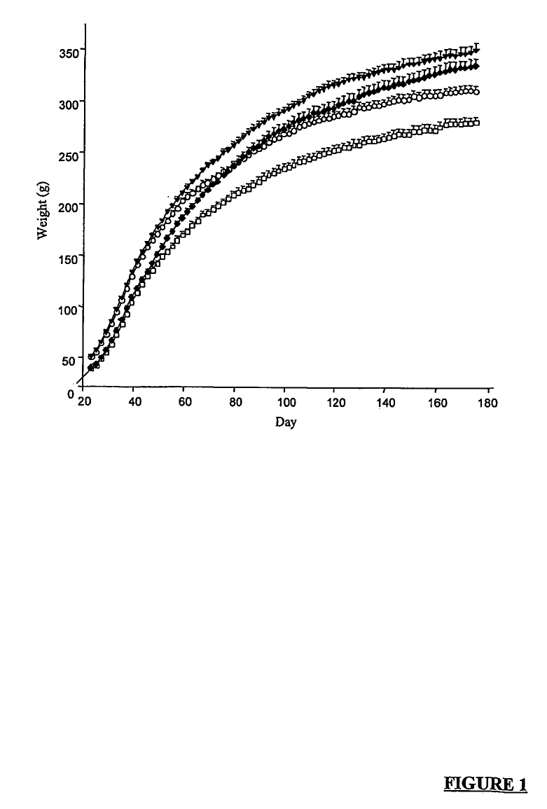

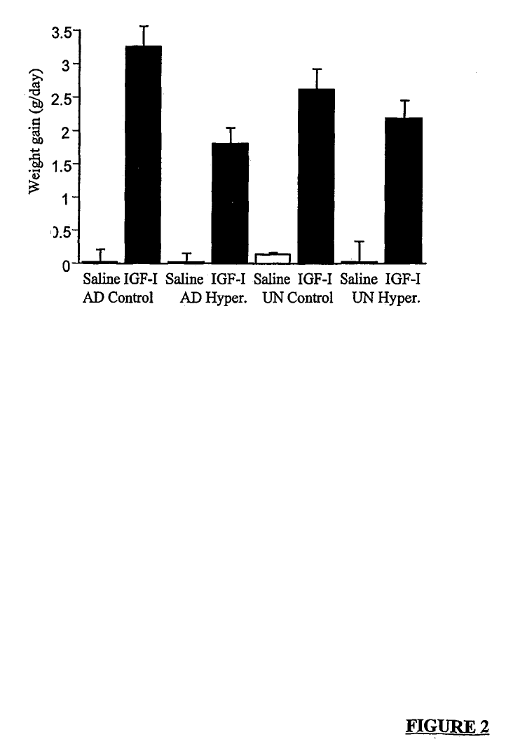

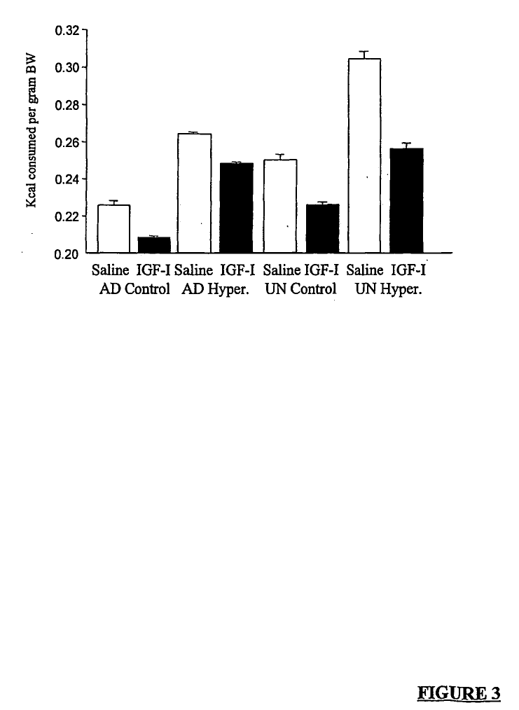

Maternal undernutrition resulted in fetal growth retardation reflected by significantly decreased body weight at birth in the offspring from UN dams (UN 4.02±0.03 g, AD 6.13±0.04 g, p<0.001). Litter size was not significantly different between the two groups (AD 11.7±1.93, UN 11.2±2.03). From birth until weaning at day 22, body weights remained significantly lower in the UN offspring (AD 51.5±0.6 g,UN 37.8±0.9 g). Total body weights on each diet remained significantly lower (p<0.0001) in UN offspring for the remainder of the study. Hypercaloric nutrition during postnatal life resulted in significantly (p<0.0001) increased body weights compared to control fed animals and by postnatal day 100 UN animals fed hypercalorically showed apparent catch-up growth to match the body weight of AD animals fed the control diet (FIG. 1). Body weight gain was increased in all IGF-I treated animals (FIG. 2) and no difference in growth response was observed between AD and UN offspring. However, daily weight gain was significantly reduced in animals treated with IGF-I on hypercaloric nutrition as reflected by the significant (p<0.05) diet×IGF-I interaction. UN offspring were shorter than AD offspring in each treatment group and nose-anus lengths were significantly (p<0.05) increased in all IGF-I treated animals (Table 2). UN animals showed a significantly higher food intake on both diets compared to AD animals. Food intake was reduced (p<0.005) in all IGF-I treated offspring (FIG. 3). A significant statistical interaction was observed between programming and IGF-I treatment whereby reduction in food intake was more pronounced in UN animals following IGF-I treatment (p<0.005).

Prior to onset of IGF-I therapy, SBP was markedly elevated (p<0.0001) in UN offspring on the control diet compared to AD offspring. The programming effect on hypertension was markedly amplified by postnatal exposure to hypercaloric nutrition (Table 1). SBP was significantly reduced with IGF-I therapy in UN animals and in the group of AD offspring which had elevated blood pressure as a result of postnatal hypercaloric nutrition (FIG. 3).

TABLE 1
Systolic blood pressure (SBP) (mmHg) prior to onset of IGF-I therapy.
Data analysed by two-way ANOVA. Data is mean ± SEM with n = 12
animals per group. There were no significant statistical interactions.

| AD Control | UN control | AD hypercaloric | UN hypercaloric |
|---|---|---|---|
| (mmHg) | (mmHg) | (mmHg) | (mmHg) |
| 121.84 ± 1.67 | 140.47 ± 2.122 | 140.04 ± 2.63 | 148.43 ± 1.59 |

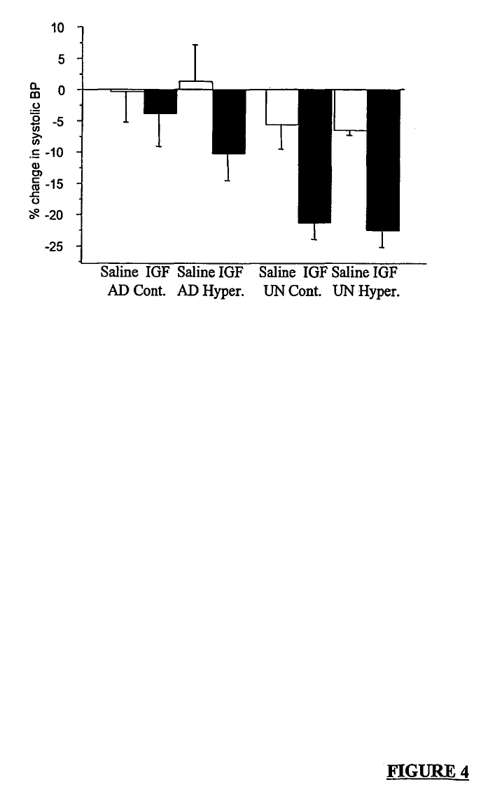

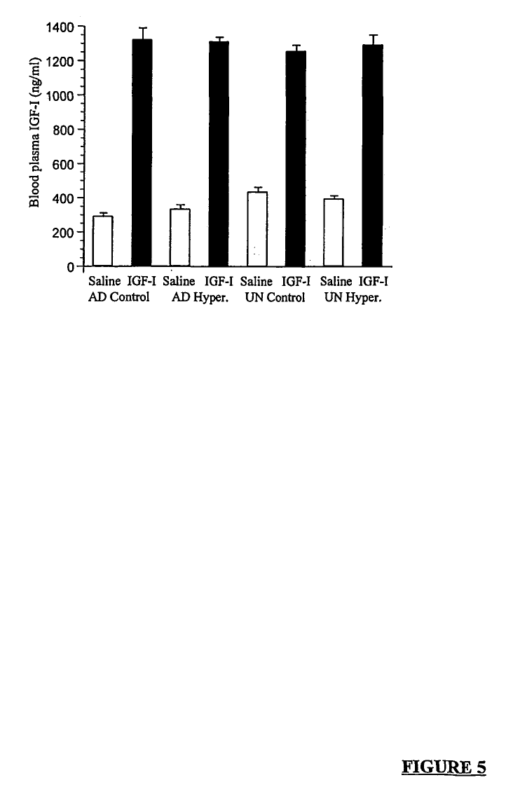

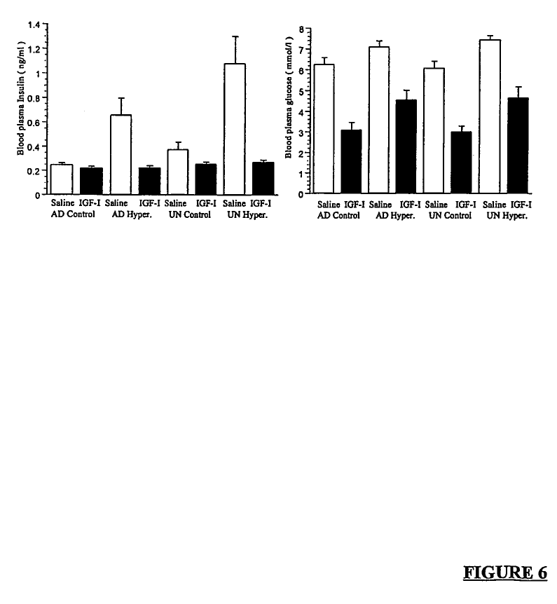

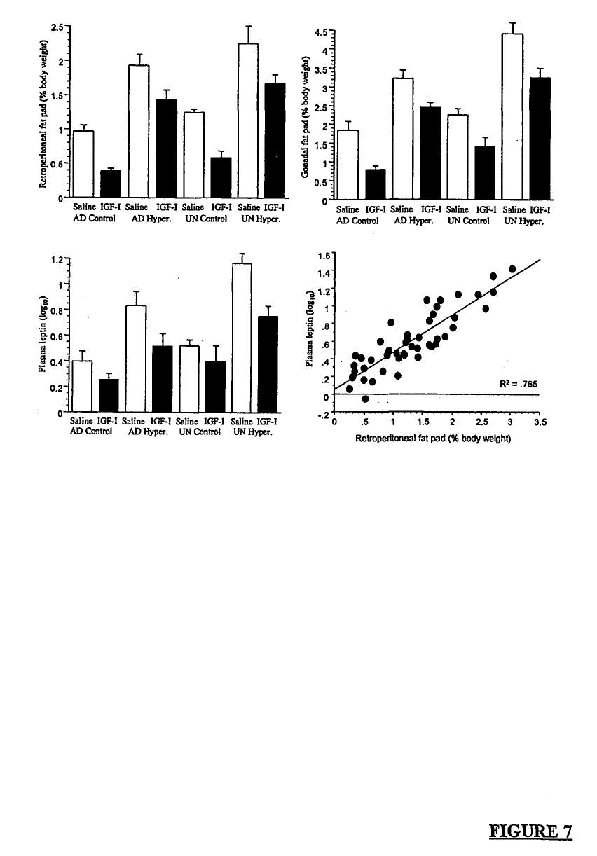

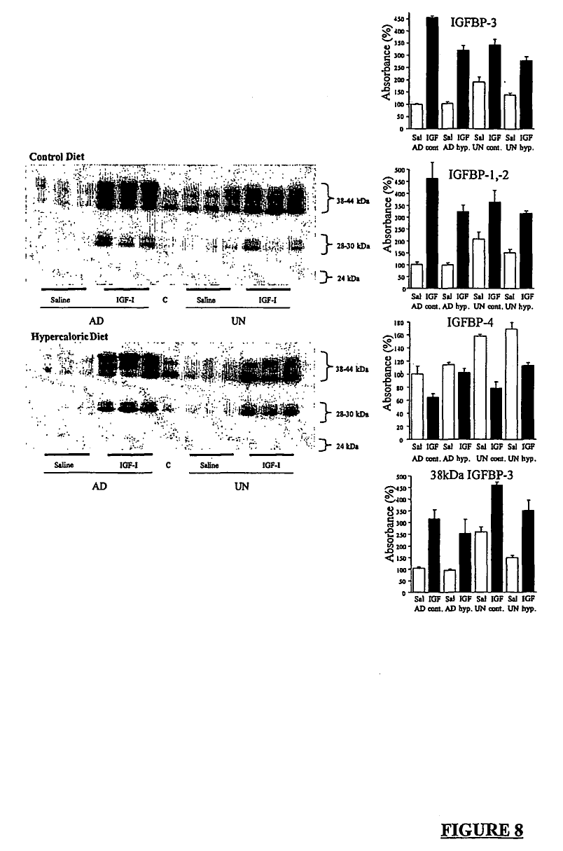

Blood plasma IGF-I concentrations were markedly increased (p<0.0001) in all IGF-treated offspring (FIG. 4). The rise in plasma IGF-I concentrations following IGF-I treatment was less in UN animals on both diets compared to AD animals (programming/diet interaction p<0.05). Fasting plasma insulin levels were higher (p<0.05) in UN offspring and were further elevated by hypercaloric nutrition (p<0.0005). Treatment with IGF-I significantly lowered insulin concentrations (p<0.005) in all offspring; this effect was most marked in the animals on hypercaloric nutrition (IGF-I treatment×diet interaction p<0.005, FIG. 5). Plasma glucose was not different between AD and UN offspring but was increased (p<0.0001) by hypercaloric nutrition. IGF-I treated animals showed markedly reduced plasma glucose concentrations (p<0.0001)(FIG. 5). Plasma leptin concentrations were higher (p<0.005) in UN offspring and were increased (p<0.0001) by hypercaloric diet. IGF-I treatment significantly lowered plasma leptin concentrations (p<0.0005). As observed with insulin, there was a strong diet-IGF-I treatment interaction (p<0.005, FIG. 7) with plasma leptin levels being most markedly reduced in offspring fed hypercalorically. Regression analysis revealed a strong positive relationship between plasma leptin and fasting insulin concentrations (r²=0.75, p<0.0001). Retroperitoneal and gonadal fat pads were significantly larger in UN offspring (p<0.05) and were further increased by hypercaloric nutrition in both AD and UN offspring (p<0.0001). Treatment with IGF-I significantly reduced fat pad mass in all treated animals (p<0.0001, FIG. 7). Regression analysis showed a highly significant positive relationship between fat mass and fasting plasma leptin (r²=0.78, p<0.001).

TABLE 2
Body weight, length and tissue weights of AD and UN offspring (age 190 ± 5 days) following 14 days treatment with IGF-I. Data
analysed by three-way factorial ANOVA followed by Bonferroni comparison. n = 6 animals per group, data are mean ± SEM.

| | | Body | | | | | | Nose-Anus |
|---|---|---|---|---|---|---|---|---|
| Group | Tx | weight (g) | Heart | Liver | Kidney | Spleen | Adrenal | (mm) |
| AD Control diet | CBS | 284 ± 5.3 | 0.38 ± 0.01 | 2.70 ± 0.07 | 0.80 ± 0.03 | 0.24 ± 0.01 | 0.028 ± 0.001 | 209 ± 2.3 |
| | IGF-I | 322 ± 3.8 | 0.40 ± 0.01 | 2.63 ± 0.03 | 0.93 ± 0.02 | 0.38 ± 0.01 | 0.036 ± 0.002 | 212 ± 2.1 |
| AD Hypercaloric diet | CBS | 341 ± 6.7 | 0.35 ± 0.01 | 2.66 ± 0.10 | 0.74 ± 0.02 | 0.21 ± 0.01 | 0.025 ± 0.001 | 208 ± 2.6 |
| | IGF-I | 374 ± 9.6 | 0.37 ± 0.01 | 2.50 ± 0.06 | 0.83 ± 0.02 | 0.38 ± 0.01 | 0.030 ± 0.002 | 217 ± 1.7 |
| UN Control diet | CBS | 258 ± 6.1 | 0.38 ± 0.01 | 2.57 ± 0.06 | 0.76 ± 0.02 | 0.25 ± 0.02 | 0.028 ± 0.001 | 195 ± 3.7 |
| | IGF-I | 297 ± 6.7 | 0.41 ± 0.01 | 2.50 ± 0.04 | 0.83 ± 0.03 | 0.36 ± 0.02 | 0.033 ± 0.001 | 203 ± 2.8 |
| UN Hypercaloric diet | CBS | 339 ± 8.9 | 0.33 ± 0.01 | 2.72 ± 0.04 | 0.68 ± 0.02 | 0.21 ± 0.01 | 0.025 ± 0.001 | 203 ± 1.7 |
| | IGF-I | 375 ± 11.4 | 0.36 ± 0.02 | 2.45 ± 0.04 | 0.73 ± 0.03 | 0.40 ± 0.05 | 0.028 ± 0.002 | 212 ± 4.7 |
| Programming effect | | p < 0.05 | NS | NS | p < 0.0001 | NS | NS | p < 0.0005 |
| IGF-I effect | | p < 0.0001 | p < 0.05 | p < 0.005 | p < 0.0001 | p < 0.0001 | p < 0.0001 | p < 0.05 |
| Diet effect | | p < 0.0001 | p < 0.0001 | NS | p < 0.0001 | NS | p < 0.0005 | p < 0.005 |
| Interactions | | | | | | | | |
| Programming × Diet | | p < 0.05 | NS | NS | NS | NS | NS | NS |
| Programming × IGF-I | | NS | NS | NS | NS | NS | NS | NS |
| Diet × IGF-I | | NS | NS | NS | NS | NS | NS | NS |
| Programming × diet × | | NS | NS | NS | NS | NS | NS | NS |
| IGF-I | | | | | | | | |

Kidney weight was significantly (p<0.0001) reduced in UN offspring (Table 2). AD and UN offspring fed hypercalorically had relatively lighter kidneys (p<0.0001). Treatment with IGF-I significantly increased kidney weight (p<0.0001). Heart weight was not different between AD and UN offspring but was reduced relative to body weight in animals fed hypercaloric nutrition. IGF-I treatment caused an increase in heart weight in all treated. animals (p<0.05). Liver weight was not different between AD and UN offspring and were not affected by diet. IGF-I treated animals had lighter livers relative to body weight compared to saline controls (p<0.005). Spleen weight was not different between AD and UN offspring and was not altered by diet. However, treatment with IGF-I caused a significant increase in spleen weight in AD and UN treated animals (p<0.0001). Relative brain weight in UN offspring was reduced as compared to AD offspring and was lighter relative to body weight (p<0.0001) in animals fed hypercalorically and/or treated with IGF-I. Adrenal weight was not different between UN and AD animals but was significantly (p<0.0001) increased with IGF-I treatment (Table 2).

Plasma free fatty acid concentrations were reduced in hypercalorically fed animals (p<0.005, Table 3) but there was no effect of programming or IGF-I treatment. Plasma urea concentrations were markedly lower in UN offspring (p<0.05, Table 3) and were decreased in all hypercalorically fed offspring (p<0.0001). Treatment with IGF-I caused a significant reduction (p<0.0001) in urea concentrations in all treated offspring. Plasma creatinine levels were not different between AD and UN offspring and were unaffected by diet. Treatment with IGF-I lowered (p<0.0001) creatinine concentrations in all treated animals (Table 3).

TABLE 3
Blood biochemistry analysis of AD and UN offspring (age 190 ± 5 days) following 14 days treatment with IGF-I. Data analysed by three-way
factorial ANOVA followed by Bonferroni comparison. n = 6 animals per group, data are mean ± SEM.

| | | Urea | Creatinine | Albumin | Magnesium | Calcium | Potassium | FFA | ALT | Bilirubin |
|---|---|---|---|---|---|---|---|---|---|---|
| Group | Tx | mmol/l | mmol/l | g/l | mmol/l | mmol/l | mmol/l | mmol/l | mmol/l | mmol/l |
| AD | CBS | 6.42 ± 0.12 | 0.058 ± 0.003 | 31.0 ± 1.03 | 0.815 ± 0.02 | 2.63 ± 0.03 | 6.83 ± 0.05 | 1.169 ± 0.13 | 30.7 ± 2.1 | 5.67 ± 0.5 |
| Control diet | IGF-I | 4.27 ± 0.25 | 0.047 ± 0.002 | 29.6 ± 0.49 | 0.945 ± 0.03 | 2.64 ± 0.04 | 7.02 ± 0.29 | 1.118 ± 0.13 | 54.2 ± 7.6 | 7.33 ± 0.4 |
| AD | CBS | 5.12 ± 0.35 | 0.057 ± 0.002 | 33.3 ± 0.42 | 0.898 ± 0.01 | 2.65 ± 0.04 | 6.88 ± 0.18 | 0.923 ± 0.06 | 35.6 ± 3.6 | 7.33 ± 0.6 |
| Hypercaloric | IGF-I | 3.02 ± 0.21 | 0.050 ± 0.002 | 29.6 ± 1.20 | 0.996 ± 0.04 | 2.61 ± 0.03 | 6.38 ± 0.30 | 0.945 ± 0.08 | 51.8 ± 2.1 | 7.40 ± 1.2 |
| diet | | | | | | | | | | |
| UN | CBS | 5.88 ± 0.42 | 0.057 ± 0.002 | 28.0 ± 0.52 | 0.855 ± 0.01 | 2.67 ± 0.03 | 6.75 ± 0.45 | 1.013 ± 0.13 | 39.6 ± 7.6 | 7.30 ± 0.7 |
| Control diet | IGF-I | 3.72 ± 0.29 | 0.050 ± 0.001 | 31.0 ± 1.06 | 1.01 ± 0.04 | 2.81 ± 0.08 | 7.45 ± 0.21 | 1.187 ± 0.16 | 55.8 ± 3.5 | 7.67 ± 1.4 |
| UN | CBS | 4.53 ± 0.12 | 0.055 ± 0.002 | 30.2 ± 0.60 | 0.867 ± 0.01 | 2.70 ± 0.02 | 6.40 ± 0.19 | 0.805 ± 0.07 | 34.6 ± 0.8 | 5.83 ± 0. |
| Hypercaloric | IGF-I | 3.03 ± 0.26 | 0.049 ± 0.001 | 28.2 ± 0.60 | 0.92 ± 0.02 | 2.68 ± 0.02 | 6.35 ± 0.36 | 0.889 ± 0.04 | 45.8 ± 3.0 | 5.83 ± 0.6 |
| diet | | | | | | | | | | |
| Program- | | P < 0.05 | NS | p < 0.05 | NS | p < 0.05 | NS | NS | NS | NS |
| ming effect | | | | | | | | | | |
| IGF-I effect | | p < 0.0001 | p < 0.0001 | NS | p < 0.0001 | NS | NS | NS | p < 0.0001 | NS |
| Diet effect | | p < 0.0001 | NS | NS | NS | NS | p < 0.05 | p < 0.005 | NS | NS |
| Interactions | | | | | | | | | | |
| Program- | | NS | NS | NS | P < 0.05 | NS | NS | NS | NS | p < 0.05 |
| ming × Diet | | | | | | | | | | |
| Program- | | NS | NS | p < 0.05 | NS | NS | NS | NS | NS | NS |
| ming × | | | | | | | | | | |
| IGF-I | | | | | | | | | | |
| Diet × IGF-I | | NS | NS | p < 0.005 | NS | NS | NS | NS | NS | NS |
| Program- | | NS | NS | NS | NS | NS | NS | NS | NS | NS |
| ming × | | | | | | | | | | |
| diet × IGF-I | | | | | | | | | | |

Alanine aminotransferase (ALT) concentrations were significantly increased (p<0.0001) in IGF-I treated offspring but were not different between AD or UN offspring and were unaltered by hypercaloric nutrition (Table 3). Albumin concentrations were significantly (p<0.05) lower in UN offspring but there was no effect of diet or treatment. Calcium levels were higher (p<0.05) in UN offspring but there was no effect of diet or tieatment. Plasma magnesium concentrations were markedly increased (p<0.0001) with IGF-I treatment but were unaffected by diet and were not different between AD and UN offspring (Table 3).

Plasma IGFBPs were analysed using nomenclature previously described (1, 26). The 38-44 kDa, 28-30 kDa and 24 kDa bands represent IGFBP-3, IGFBP-1,-2 and IGFBP-4 respectively.

Analysis of plasma IGFBPs revealed that basal levels of the different IGFBPs were elevated in UN offspring compared to AD offspring. IGF-I treatment resulted in a 3- to 5-fold increase (p<0.001) in IGFBP-3 in all IGF-I treated animals (FIG. 8). However, there was a diminished up-regulation of IGFBP-3 in UN animals indicated by a significant (p<0.0001) programming×IGF-I treatment interaction (p<0.0001). Hypercaloric nutrition significantly (p<0.0001) reduced the IGFBP-3 band compared to animals on the control diet and diminished (p<0.0001) the up-regulation of IGFBP-3 following IGF-I treatment which was further amplified in UN animals with a significant (p<0.05) programming×diet×IGF-I treatment interaction. Interestingly although in UN animals the combined 38-44 kDa IGFBP-3 band showed impaired up-regulation following IGF-I treatment, analysis of the 38 kDa band alone showed a marked increase in this band in UN animals indicating a differential pattern of up-regulation in UN animals.

Treatment with IGF-I significantly (p<0.0001) increased (2 to 5 fold) the 28-30 kDa bands representing IGFBP-1 and 2 and as observed with IGFBP-3 there was a diminished up-regulation of IGFBP-3 following IGF-I treatment in UN animals compared to AD animals (p<0.05). Similarly, hypercaloric nutrition significantly reduced the increase in IGFBP-1 and 2 following IGF-I treatment.

The 24 kDa band representing IGFBP4 was significantly elevated in all UN animals (p<0.0001) and was further amplified in all animals fed hypercalorically (p<0.0001). In an opposing pattern to what was observed with IGFBP-1 to -3, a significant (p<0.0001) down-regulation of IGFBP4 was observed following IGF-I treatment. A significant (p<0.001) programming×IGF-I treatment interaction revealed that IGFBP4 was more markedly down-regulated in UN animals following IGF-I treatment compared to AD animals. A significant diet×IGF-I treatment interaction was observed with IGF-I treatment resulting in a lesser reduction in IGFBP-4 in hypercalorically fed animals compared to those fed the control diet.

Thus, IGF-I treatment leads to a significant increase in body length, a marked reduction in food intake, decreased body fat mass and normalisation of blood pressure. Further endocrine responses include normalisation of fasting insulin and glucose concentrations and a major reduction in plasma leptin concentrations. The observation of a reduction in food intake despite the plasma leptin and insulin lowering effects of IGF-I invites a novel interpretation of IGF-I action. Firstly, IGF-I treatment may abolish the programming-induced leptin resistance at the leptin-hypothalamic circuitry and at the pancreatic adipoinsular feedback system. Secondly, IGF-I treatment may also ameliorate insulin resistance, both centrally and peripherally.

UN animals were hyperphagic on both postnatal diets compared to AD animals confirming earlier observations(3). However, the significant decrease in plasma leptin concentrations following IGF-I treatment was associated with a decrease in food intake. The decrease was more pronounced in offspring that were programmed to become obese and hyperphagic in adult life and may explain the reduced body weight gain observed in IGF-I treated offspring fed hypercaloric nutrition. The reduced food intake following IGF-I treatment may be the result of the anorectic effect of IGF-I via its insulin-sensitizing effects and reduction of chronic hyperinsulinemia. Food intake was most markedly reduced in programmed animals fed hypercaloric nutrition; the same animals that showed the most marked decrease and normalisation of fasting insulin concentrations and normoglycemia following IGF-I treatment.

Our data on the lipolytic effect of IGP-I support that of others (27-29) and suggest that the effects of prolonged IGF-I treatment on adipose tissue are not insulin-like as reflected by increased lipolysis and decreased body fat mass. IGF-I treatment may reduce body fat mass via an inhibition of the lipogenic capacity of adipocytes and reduction of lipogenesis in adipose tissue via inhibition of insulin secretion. IGF-I showed marked lipolytic effects with retroperitoneal and gonadal fat pad mass being markedly reduced concomitant with a significant decrease in fasting plasma leptin concentrations.

An endocrine feedback loop between insulin and leptin, the adipoinsular axis, has recently been proposed (30) and it has further been suggested that conditions of increasing adiposity and prolonged elevation of plasma leptin concentration result in a dysregulation of the adipoinsular axis (31, 32). Our data add support to this concept and suggest that the interaction between the leptin and insulin signalling networks is disrupted as a result of fetal programming and fulrther exacerbated by postnatal hypercaloric nutrition. Such a dysregulation of the adipoinsular axis may contribute to some of the alterations in the effects of insulin action that are involved in the progression to insulin resistance and adipogenic diabetes. Insulin receptor substrates 1 and 2 (IRS-1 and IRS-2) co-ordinate essential effects of insulin/IGF upon peripheral metabolism and beta cell function. Recent evidence suggests that impaired IRS-1 expression and downstream signalling events in adipocytes in response to insulin are associated with insulin resistance and the pentad of hypertension, hyperinsulinemia, dyslipidemia, obesity, and cardiovascular disease, known as Syndrome X (33). Furthermore, chronic hyperinsulinemia downregulates the mRNA for IRS-2, an essential component of the hepatic insulin signalling pathway, thereby exacerbating the insulin resistant state (34). Leptin can modify insulin-induced changes in gene expression in vivo (35) and the high concentrations of leptin required to obtain inhibition of signal transduction reflect the hyperleptinemia associated with obesity in the insulin resistant state (36). Furthermore, IRS-2 plays a special role in carbohydrate metabolism through mediation of both peripheral insulin action and pancreatic beta cell function. Pancreatic beta cells express little or no insulin receptor but large amounts of type 1 IGF-1 receptor which are proposed to promote islet and beta cell growth and survival, especially to compensate for peripheral insulin resistance (37). IGF-I has been shown to inhibit insulin secretion from beta cells through an IGF-1 receptor-mediated pathway (38, 39) and the IGF-I-IRS-2 signalling pathway has been proposed to be critical for postnatal beta cell function (37). On the basis of this information and our results, treatment with IGF-I may restore some of the cross talk between leptin and insulin via a differential modification of the metabolic and mitotic effects of insulin exerted through IRS-1 and IRS-2 and the downstream signalling events they activate.

The highly significant increase in kidney weight with IGF-I treatment may be an important factor in the reduction of systolic blood pressure via changes in renal plasma flow and glomerular filtration rate. IGF-I treatment may reduce blood pressure by down-regulating the local renin-angiotensin system (RAS). Importantly, IGF-I treatment only reduced systolic blood pressure in animals that were hypertensive as a result of fetal programming and postnatal hypercaloric nutrition, while systolic blood pressure in normotensive animals remained unaltered.

This effect on blood pressure may result from improved insulin sensitivity and glycemic control in conjunction with the known vasodilatory effects of exogenous IGF-I. It may also, or alternatively involve the effects of IGF-I on leptin, which is believed to play an important role in the pathogenesis of obesity-related hypertension (40, 41).

The effect of IGF-I treatment on improving insulin sensitivity and ameliorating the postnatal pathophysiology following fetal programming may be mediated by circulating IGFBPs. As IGF actions are modified by IGFBPs, the induction of binding proteins by IGF-I may act as a regulator of IGF-I in target tissues. We investigated the circulating levels of IGFBPs to examine whether fetal programming results in a differential expression of IGFBPs and whether such expression is altered by postnatal hypercaloric nutrition. The mechanism underlying the preferential up-regulation of the 38 kDa IGFBP-3 band in UN animals following IGF-I treatment is unclear.

Fetal programming resulted in a significant elevation in IGFBP-4 concentrations which were markedly amplified by postnatal hypercaloric nutrition. Treatment with IGF-I resulted in a significant decrease in circulating IGFBP-4 in all treated animals and, moreover, IGF-I treatment was more effective in reducing IGFBP4 concentrations in those animals that had become obese as a result of fetal programming and hypercaloric nutrition. Activation of IGFBP-4 proteases by exogenous IGF-I may result in the degradation and inactivation of IGFBP-4. Our data show a reduced up-regulation of IGFBP-3 with IGF-I treatment following fetal programming concomitant with a more pronounced decrease in serum IGFBP-4 concentrations. Thus, IGFBP-4 induced restraint on IGF-I activity at the tissue level may be reduced and could partially explain the amelioration of programming and diet-induced pathophysiology observed. To date, these data are the first to report an impaired and differential up-regulation of IGFBPs following IGF-I treatment in adults which have been subjected to fetal programming; an impairment which is significantly altered by exposure to hypercaloric nutrition postnatally.

Discussion

As we have stated previously, our animal model displays a phenotype that closely resembles that described in the clinical setting as the metabolic syndrome or “Syndrome X” (33). Syndrome X is a multifaceted syndrome characterized by the clustering of insulin resistance and hyperinsulinemia, and is often associated with hypertension, obesity, glucose intolerance and type 2 diabetes (42). Despite the suggestion that insulin itself mediates the clinical linkage, the specific mechanisms underlying this syndrome remain poorly understood. Our data show that IGF-I therapy alleviates insulin resistance, hyperleptinemia and hypertension and may restore functional feedback between insulin and leptin following perturbations in the adipoinsular axis as a result of fetal programming. IGF-I therapy may also ameliorate obesity, hyperphagia and hypertension by differential regulation of downstream signalling networks via the IRS, RAS and IGF-I receptor signalling pathways by independent and complementary mechanisms.

Of particular clinical benefit is the potential use of IGF-I, analogues or ligands, in individuals who have been exposed to fetal programming but are otherwise essentially healthy (ie. prior to the development of the consequences of fetal programming, such as hypertension, obesity, hyperphagia, diabetes, and other metabolic disorders). Indeed, it is conceivable that such therapy may reverse the effects of fetal programming.

The application of this invention to humans and other animals suffering from fetal programing due to the effects of undernutrition and IUGR will allow for prophylactic, and also direct, treatment regimes. As is readily apparent, review of maternal health (as is discussed herein) will allow clear indication of individuals who are likely to suffer from conditions associated with fetal programming later in life. Treatment, following identification of at risk individuals, is a simple and relatively cheap matter using accepted techniques. In animals other than humans similar opportunities arise. Application to production animals (sheep, cows, deer etc.) suffering from the effects of drought or similar environmental conditions, could mitigate the ongoing effects (e.g. on. body weight, health etc) of the next generation of animals.

Where in the foregoing description reference has been made to specific components or integers of the invention having known equivalents then such equivalents are herein incorporated as if individually set forth.

Although this invention has been described by way of example and with reference to possible embodiments thereof it is to be understood that modifications or improvements may be made thereto without departing from the scope or spirit of the invention as detailed in the appended claims.

### REFERENCES

1. Woodall S M, Breier B H, Johnston B M, Gluckman P D 1996 A model of intrauterine growth retardation caused by chronic matemal undernutrition in the rat: effects on the somatotropic axis and postnatal growth. J Endocrinol 150:231-242

2. Woodall S M, Johnston B M, Breier B H, Gluckman P D 1996 Chronic maternal undernutrition in the rat leads to delayed postnatal growth and elevated blood pressure of offspring. Pediatr Res 40:438-443

3. Vickers M H, Breier B H, Cutfield W S, Hofinan P L, Gluckman P D 2000 Fetal origins of hyperphagia, obesity and hypertension and its postnatal amplification by hypercaloric nutrition. Am J Physiol 279:E83-E87

4. Jackson A A, Langley-Evans S C, McCarthy H D 1996 Nutritional influences in early life upon obesity and body proportions. Ciba Foundation Symposium 201:118-129

5. Ravelli A C, van der Meulen J H, Osmond C, Barker D J, Bleker O P 1999 Obesity at the age of 50 y in men and women exposed to famine prenatally. Am J Clin Nutr 70:811-816

6. Anguita R M, Sigulem D M, Sawaya A L 1993 Intrauterine food restriction is associated with obesity in young rats. J Nutr 123:1421-1428

7. Jones A P, Pothos E N, Rada P, Olster D H, Hoebel B G 1995 Matemal hormonal manipulations in rats cause obesity and increase medial hypothalamic norepinephrine release in male offspring. Brain Res Developmental Brain Research. 88:127-131

8. Baker J, Liu J P, Robertson E J, Efstratiadis A 1993 Role of insulin-like growth factors in embryonic and postnatal growth. Cell 75:73-82

9. Powell-Braxton L, Hollingshead P, Warburton C, Dowd M, Pitts-Meek S, Dalton D, Gillett N, Stewart T A 1993 IGF-I is required for normal embryonic growth in mice. Genes & Development 7:2609-2617

10. Muaku S M, Thissen J P, Gerard G, Ketelslegers J M, Maiter 1997 Postnatal catch-up growth induced by growth hormone and insulin-like growth factor-I in rats with intrauterine growth retardation caused by maternal protein malnutrition. Pediatr Res 42:370-377

11. Froesch E R, Bianda T, Hussain M A 1996 Insulin-like growth factor-I in the therapy of non-insulin-dependent diabetes mellitus and insulin resistance. Diabetes & Metabolism 22:261-267

12. Dunger D B, Acerini C L 1997 Does recombinant human insulin-like growth factor-1 have a role in the treatment of diabetes?. Diabetic Medicine 14:723-731

13. Boni-Schnetzler M, Hauri C, Zapf J 1999 Leptin is suppressed during infusion of recombinant human insulin-like growth factor I (rhIGF I) in normal rats. Diabetologia 42:160-166

14. Zenobi P D, Jaeggi Groisman S E, Riesen W F, Roder M E, Froesch E R 1992 Insulin-like growth factor-I improves glucose and lipid metabolism in type 2 diabetes mellitus. J Clin Invest 90:2234-2241

15. Tomas F M, Chandler C S, Coyle P, Bourgeois C S, Burgoyne J L, Rofe A M 1994 Effects of insulin and insulin-like growth factors on protein and energy metabolism in tumour-bearing rats. Biochem J 301:769-775

16. Donath M Y, Sutsch G, Yan X W, Piva B, Brunner H P, Glatz, Zapf J, Foflath F, Froesch E R, Kiowski W 1998 Acute cardiovascular effects of insulin-like growth factor I in patients with chronic heart failure. J Clin Endocrinol Metab 83:3177-3183

17. Delafontaine P 1995 Insulin-like growth factor I and its binding proteins in the cardiovascular system. Cardiovasc Res 30:825-834

18. Blum W F, Ranke M B 1990 Use of insulin-like growth factor-binding protein 3 for the evaluation of growth disorders. Horm Res 34(suppl 1):31-37

19. Jones J I, Clemmons D R 1995 Insulin-like growth factors and their binding proteins: Biological actions. Endocr Rev 16:3-34

20. Unterman T G, Lascon R, Gotway M B, Oehler D T, Gounis A, Simmons R A, Ogata E S 1990 Circulating levels of insuin-like growth factor binding protein-1 (IGFBP-1) and hepatic mRNA are increased in the small for gestational age (SGA) fetal rat. Endocrinology 127:2035-2037

21. Tapanainen P J, Bang P, Wilson K, Unterman T G, Vreman H J, Rosenfeld R G 1994 Maternal hypoxia as a model for intrauterine growth retardation: effects on insulin-like growth factors and their binding proteins. Pediatr Res 36:152-158

22. Blum W F, Breier B H 1994 Radioimmunoassays for IGFs and IGFBPs. Growth Regulation 4:11-19

23. Breier B H, Vickers M H, Gravance C G, Casey P J 1996 Growth hormone (GH) therapy markedly increases the motility of spermatozoa and the concentration of insulin-like growth factor-I in seminal vesicle fluid in the male GH-deficient dwarf rat. Endocrinology 137:4061-4064

24. Hossenlop P, Seurin D, Segovia-Quinson B, Hardouin S, Binoux M 1986 Analysis of serum insulin-like growth factor binding proteins using Western-ligand blotting: use of the method for titration of the binding proteins and competitive binding studies. Ann Biochem 154:138-143

25. Gallaher B W, Breier B H, Oliver M H, Harding J E, Gluckman P D 1992 Ontogenic differences in the nutritional regulation of circulating IGF binding proteins in sheep plasma. Acta Endocrinol Copenh 126:49-54

26. Gargosky S E, Tapainen P, Rosenfeld R G 1994 Administration of growth hormone (GH), but not insulin-like growth factor-I (IGF-I), by continuous infusion can induce the formation of the 150-kilodalton IGF-binding protein-3 complex in GH-deficient rats. Endocrinology 134:2267-2276

27. Guler H -P, Zapf J, Scheiwiller E, Froesch E R 1988 Recombinant human insulin-like growth factor I stimulates growth and has distinct effects on organ size in hypophysectomised rats. Proc Natl Acad Sci USA 85:4889-4893

28. Tomas F M, Knowles SE, Owens P C, Chandler C S, Francis G L, Ballard F J 1993 Insulin-like growth factor-I and more potent variants restore growth of diabetic rats without inducing all characteristic insulin effects. Biochem J 291:781-786

29. Frick F, Oscarsson J, Vikman-Adolfsson K, Ottosson M, Yoshida N, Eden S 2000 Different effects of IGF-I on insulin-stimulated glucose uptake in adipose tissue and skeletal muscle. American Journal of Physiology—Endocrinology & Metabolism 278:E729-E737

30. Kieffer T J, Heller R S, Habener J F 1996 Leptin receptors expressed on pancreatic beta-cells. Biochem Biophys Res Commun 224:522-527

31. Seufert J, Kieffer T J, Leech C A, Holz G G, Moritz W, Ricordi C, Habener J F 1999 Leptin suppression of insulin secretion and gene expression in human pancreatic islets: implications for the development of adipogenic diabetes mellitus. J Clin Endocrinol Metab 84:670-676

32. Kieffer T J, Habener J F 2000 The adipoinsular axis: effects of leptin on pancreatic beta-cells. Am J Physiol 278:E1-E14

33. Reaven G M 1993 Role of insulin resistance in human disease (syndrome X): an expanded definition. Annual Review of Medicine 44:121-131

34. Shimomura I, Matsuda M, Hammer R E, Bashmakov Y, Brown M S, Goldstein J L 2000 Decreased IRS-2 and increased SREBP-1c lead to mixed insulin resistance and sensitivity in livers of lipodystrophic and ob/ob mice. Mol Cell Biochem 6:77-86

35. Szanto I, Kahn C R 2000 Selective interaction between leptin and insulin signalling pathways in a hepatic cell line. Proc Natl Acad Sci USA 97:2355-2360

36. Cohen B, Novick D, Rubinstein M 1996 Modulation of insulin activities by leptin. Science 274:1185-1188

37. Withers D J, White M F 2000 Perspective: The insulin signalling system—a common link in the pathogenesis of type 2 diabetes. Endocrinology 141:1917-1921

38. Zhao A Z, Zhao H, Teague J, Fujimoto W, Beavo J A 1997 Attenuation of insulin secretion by insulin-like growth factor 1 is mediated through activation of phosphodiesterase 3B. Proc Natl Acad Sci USA 94:3223-3228

39. Van Schravendijk C F, Heylen L, Van den Brande J L, Pipeleers D G 1990 Direct effect of insulin and insulin-like growth factor-I on the secretory activity of rat pancreatic beta cells. Diabetologia 33:649-653

40. Agata J, Masuda A, Takada M, Higashiura K, Murakami H, Miyazaki Y, Shimamoto K 1997 High plasma immunoreactive leptin level in essential hypertension. American Journal of Hypertension 10:1171-1174

41. Aizawa-Abe M, Ogawa Y, Masuzaki H, Ebihara K, Satoh N, Iwai H, Matsuoka N, Hayashi T, Hosoda K, Inoue G; Yoshimasa Y, Nakao K 2000 Pathophysiological role of leptin in obesity-related hypertension. J Clin Invest 105:1243-1252

42. Resnick L M 1993 Ionic basis of hypertension, insulin resistance, vascular disease, and related disorders. The mechanism of “syndrome X”. American Journal of Hypertension 6:123S-134S

1. A method of ameliorating or preventing the consequences of fetal programming in an otherwise normal mammal, including the administration to the mammal of an effective amount of insulin-like growth factor (IGF-I), an analogue thereof, or a functionally equivalent ligand.

2. A method of ameliorating or preventing the consequences of fetal programming in a mammal, including the steps of:
identifying a mammal exposed to fetal programming, and
treating said mammal with an effective amount of IGF-I, an analogue thereof, or a functionally equivalent ligand.

3. The method according to claim 1 or claim 2 wherein the mammal is a human being.

4. The method according to any one of claims 1 to 3 wherein the mammal has no physiological symptoms and/or outward signs of conditions resulting from the fetal programming.

5. The method according to any one of the preceding claims wherein the mammal exposed to fetal programming is identified from a review of maternal history during pregnancy.

6. The method according to any one of the preceding claims wherein the fetal programming is identified by one or more physiological or metabolic indicators selected from any one or more of maternal food deprivation, placental dysfunction, uteroplacental blood supply, intrauterine growth retardation, altered levels of IGF-1, and inter-generational effects.

7. The method according to any one of the preceding claims wherein the IGF-1, analogue thereof, or functionally equivalent ligand is encoded in a replicable vehicle.

8. The method according to any one of the preceding claims wherein the IGF-1, analogue thereof, or functionally equivalent ligand is administered by subcutaneous injection.

9. The use of an agent selected from IGF-I, an analogue thereof or a functionally equivalent ligand, in the preparation of a medicament for ameliorating or preventing the consequences of fetal programming in a mammal.

10. The use according to claim 9 wherein the agent is a replicable vehicle encoding the IGF-1 analogue thereof or functionally equivalent ligand.

11. The use according to claim 9 or claim 10 wherein the medicament is an implant.

12. The use according to claim 9 wherein the medicament is a subcutaneous injection.

13. A method of treating hyperinsulinemia or insulin resistance in a mammal exposed to fetal programming including identification of a mammal exposed to fetal programming, and administration of an effective amount of IGF-I, an analogue thereof or a functionally equivalent ligand to the mammal.

14. The method according to claim 13 wherein the IGF-1 is administered in a sterile saline or water solution.

15. The method according to claim 13 wherein the method includes administration of a replicable vehicle encoding the IGF-I, an analogue thereof or a functionally equivalent ligand.

16. A prophylactic method of treating hyperphagia, hypertension, hyperinsulinemia, dyslipidemia, obesity, insulin resistance, and cardiovascular disease in a mammal exposed to fetal programming and not exhibiting symptoms of hyperinsulinemia or insulin resistance including identifying the existence of fetal programming and administration of an effective amount of IGF-I, an analogue thereof or a functionally equivalent ligand.

17. A method of treating the consequences of fetal programming substantially as herein described.
